(19) United States
(12) Patent Application Publication
(10) Pub. No.: US 2019/0150807 A1
(43) Pub. Date: May 23, 2019

(54) SYSTEMS AND METHODS FOR EVALUATING INTERSTITIAL FLUIDS

(71) Applicant: Paul Andrew Glazer, Boston, MA (US)

(72) Inventor: Paul Andrew Glazer, Boston, MA (US)

(21) Appl. No.: 16/198,305

(22) Filed: Nov. 21, 2018

Related U.S. Application Data
(60) Provisional application No. 62/589,703, filed on Nov. 22, 2017.

(51) Int. Cl. A61B 5/145; B01L 3/00; G01N 33/94; G01N 33/76; G01N 33/569; G01N 33/84; G01N 33/68; A61B 10/00; A61B 5/00

(52) U.S. Cl. CPC A61B 5/14514; A61B 5/4845; G01N 33/94; G01N 33/76; G01N 33/5695; G01N 33/948; G01N 33/84; G01N 33/6887; A61B 10/0045; A61B 5/6833; G01N 2333/59; G01N 2333/35; G01N 2333/78; A61B 2010/008; A61B 5/14546; B01L 3/502715

(57) ABSTRACT

A transdermal sampling system includes a microfluidic assembly for retrieving and transferring at least one biomarker transdermally from the skin of a subject. The biomarker is selected from a group of biomarkers consisting of: a nicotine biomarker, β-hCG, luteinizing hormones, ESAT6; CFP10, Hsp16.3, marijuana and its derivatives, opiates, narcotics, ethanol, barbiturates, pyridinoline crosslinks, Calcitonin, FSH, estrogen, vitamin D, calcium, glial fibrillary acid protein (GFAP), microtubule-associated protein tau, SNTF-calpain cleaved N terminal fragment proteins, estradiol, P4-progesterone, troponin, lactate, CO₂ levels, and pH levels. A transdermal detector identifies and quantifies the at least one biomarker.

### CROSS-REFERENCE TO RELATED APPLICATIONS

This application claims benefit of priority to U.S. Provisional Patent Application No. 62/589,703 filed Nov. 22, 2017, which is incorporated by reference herein in its entirety.

### BACKGROUND OF THE INVENTION

### 1. Field of the Invention

The present invention relates to the field of portable biomedical and bio-molecular monitoring, remote diagnostics and connected healthcare. More specifically, the present invention relates to a method, apparatus, and system for a configurable flexible personal health monitoring and material delivery system.

### 2. Description of Related Art

Non-invasive transdermal sampling of body fluids has long been a goal in medical research. The notion that valuable diagnostic information comprising the concentrations of key biomarkers within the bloodstream could be obtained without breaching the skin has spurred many lines of research. With such technology, long-term convenient health monitoring and screening without needles or outpatient care would become a reality: diabetics could monitor blood glucose without drawing blood; markers for microbial, fungal or viral infections could be monitored; and environmental exposure to toxins could be assessed non-invasively.

Such conventional methods and systems have generally been considered satisfactory for their intended purpose. However, there is still a need in the art for improved transdermal sampling systems and methods. The present invention provides a solution for these problems.

### BRIEF DESCRIPTION OF THE DRAWINGS

So that those skilled in the art to which the subject invention appertains will readily understand how to make and use the devices and methods of the subject invention without undue experimentation, preferred embodiments thereof will be described in detail herein below with reference to certain FIGURES, wherein:

FIG. 1 is a perspective view of an exemplary embodiment of a patch constructed in accordance with embodiments of the invention, showing a transdermal detector system within the patch.

### SUMMARY OF THE INVENTION

A transdermal sampling system includes a microfluidic assembly for retrieving and transferring at least one biomarker transdermally from the skin of a subject. The biomarker is selected from a group of biomarkers consisting of: a nicotine biomarker, β-hCG, luteinizing hormones, ESAT6; CFP10, Hsp16.3, marijuana and its derivatives, opiates, narcotics, ethanol, barbiturates, pyridinoline crosslinks, Calcitonin, FSH, estrogen, vitamin D, calcium, glial fibrillary acid protein (GFAP), microtubule-associated protein tau, SNTF-calpain cleaved N terminal fragment proteins, estradiol, P4-progesterone, troponin, lactate, CO₂ levels, and pH levels. A transdermal detector identifies and quantifies the at least one biomarker.

In accordance with another aspect of the invention, a transdermal sampling includes a microfluidic assembly in the form of a patch.

### DETAILED DESCRIPTION OF THE PREFERRED EMBODIMENTS

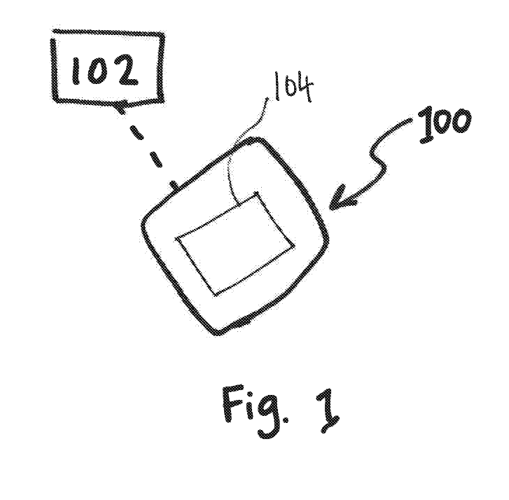

Reference will now be made to the drawings wherein like reference numerals identify similar structural features or aspects of the subject invention. For purposes of explanation and illustration, and not limitation, a perspective view of an exemplary embodiment of a patch constructed in accordance with embodiments of the invention is shown in FIG. 1 and is designated generally by reference character 100. Embodiments described herein provide a minimally invasive sampling technique, an apparatus suitable for rapid, inexpensive, unobtrusive, and pain-free monitoring of important biomedical markers and environmental toxin exposure.

As shown in FIG. 1, a medium, for example, a microfluidic assembly, evaluates the presence of and measures proteins and other substances within the interstitial fluid to identify medical conditions. The microfluidic assembly can be in the form of a band or a patch 100. Patch 100 collects and samples interstitial fluid to identify biological markers associated with certain conditions. Patch 100 is operatively connected to a transdermal detector system 104 for identifying and quantifying said at least one of the biomarkers described below. A logic module 102 or monitoring system can be used to receive and store input data from the detector, relate the input data to other data obtained from the subject relating to the condition of the subject, and display output information indicative of health and clinical state of the subject as determined from the relating of the input data to the other data. It is contemplated that the data can be transmitted to one or more other systems. It is also contemplated that the operation of the patch can be controlled by transdermal detector 104. Patch 100, detector system 104 and logic module 102 can all be constructed in accordance with U.S. Pat. Nos. 6,887,202; 7,931,592; 8,568,315; 8,364,228; 8,333,874; and U.S. Patent Publication Nos. 2014-0275895; 2014-0025000, which are all incorporated by reference herein in their entirety.

In accordance with one embodiment, patch 100 can collect interstitial fluid and measure and detect nicotine biomarkers with detector 104. The measurement is used to assess the risk of pseudoarthrosis or failure of healing of bone fractures and spinal fusion procedures. The presence of nicotine can be detected in a practitioner's office prior to an elective procedure in order to assess the pre-operative risk, or the like.

In accordance with another embodiment, interstitial fluid can be collected and measurements of β-hCG (beta human chorionic gonadotropin) can be taken. This is less invasive than convention methods of detecting pregnancy, such as blood tests and urine samples.

Additionally, patch 100 can be used as a monitoring device to detect ovulation. Patch 100 can collect interstitial fluid and detector 104 can take serial measurements of hormones, such as Estradiol, P4-progesterone, and luteinizing hormone (LH) to determine whether a woman is ovulating. A monitoring system 102 is operatively connected to patch 100 to alert a woman when ovulation is occurring. This information can be used by a woman who wants to conceive, or by a woman who wants to avoid conception. It is contemplated that monitoring system 102 can be integral to patch 100 or can be separated from patch 100, for example, in a computer, tablet or smart phone. This is more precise than conventional methods for assessing ovulation, such as detection in temperature elevation.

In accordance with another embodiment, interstitial fluid is collected through patch 100 and measurements of Tuberculosis (Tb) anitgens such as ESAT6, CFP10 and Hsp16.3. After the measurements are taken, they are used to assess whether the patient has a Tb infection through detector 104. Systems and methods in accordance with the embodiments of the present invention would provide instantaneous knowledge of prior TB exposure and infection, e.g. by using monitoring system 102. Tuberculosis is a worldwide health problem and conventional methods of detection require a skin allergy test or purified protein derivative (PPD) placement. This conventional method typically requires an evaluation of the skin area 48-72 hours after the subcutaneous injection of the allergen. It is also contemplated that patch 100 would be able to detect even latent Tb infections, where there is no clinical or radiographic or bacteriologic evidence of infection.

In accordance with another embodiment, interstitial fluid can be collected through patch 100 and the presence of marijuana and its derivatives, opiates, ethanol, barbiturates, or other narcotics are detected through detector 104. This tends to be useful in assessing patients who cannot provide a detailed medical history or who do wish to provide such details. Conventional methods to assess levels of marijuana and its derivatives, opiates, barbiturates, ethanol, or other narcotics require blood tests and breath analyses, respectfully.

In accordance with another embodiment, interstitial fluid can be collected through patch 100 and measurements of proteins such as pyridinoline crosslinks, and the like, are taken with detector 104 to assess osteoporosis or bone breakdown in a patient. It is contemplated that Calcitonin, FSH, estrogen, vitamin D, and calcium levels can be measured, monitored, and/or assessed as well. Those skilled in the art will readily appreciate that measuring, monitoring and assessing these proteins with interstitial fluids is faster and easier than conventional methods, such as urine tests.

In accordance with another embodiment, patch 100 collects interstitial fluid and measures glial fibrillary acid protein (GFAP), microtubule-associated protein tau, and SNTF-calpain cleaved N terminal fragment proteins to diagnose the mental health of patients. This assessment can be conducted quickly and efficiently in order to determine whether it is okay to allow a patient, e.g. an athlete, to continue activity. These proteins are typically expressed in blood after acute brain trauma. This is less costly and more efficient than conventional clinical evaluation for concussions, such as medical imaging.

In accordance with another embodiment, patch 100 collects interstitial fluid and measures CO₂ levels or the pH of the interstitial fluid using detector 104. This measurement is assessed and used to diagnose and prevent sleep apnea or sudden infant death syndrome (SIDS).

In accordance with another embodiment, patch 100 collects interstitial fluid and detects proteins with detector 104 to identify a patient status. This allows patch 100 to be used as patient monitoring system, for example, to assess if they have fallen and may require attention.

In accordance with another embodiment, interstitial fluid is collected through patch 100 and measurements of a protein such as troponin are taken with detector 104 to assess whether the heart muscle has been damaged, e.g. damage that occurs with a heart attack. This is less invasive an easier to administer than the traditional blood tests used for detecting and measuring troponin.

In accordance with another embodiment, interstitial fluid is collected through patch 100. Measurements of lactate are taken with detector 104 to assess whether a patient has lactic acidosis, a level of lactate that is high enough to disrupt a person's acid-base (ph) balance. This measurement can assist in determining the existence or severity of shock or congestive heart failure, and/or can assist in evaluating whether a patient has liver or kidney disease.

It is contemplated that patch 100 can use Single Molecule Array (Simoa) as part of a detector, e.g. detector 104, to measure the proteins described above. Simoa uses highly sensitive immunoassays and allows accurate measurements of candidate biomarkers in blood. Simoa uses a digital approach that uses arrays of femtoliter-sized reaction chambers that can isolate and detect single enzyme molecules and provides a 1000-fold improvement in sensitivity over conventional immunoassays.

The methods and systems of the present invention, as described above and shown in the drawings, provide for diagnostic tools with superior properties including reduced invasiveness and increased efficiency. While the apparatus and methods of the subject invention have been shown and described with reference to preferred embodiments, those skilled in the art will readily appreciate that changes and/or modifications may be made thereto without departing from the spirit and scope of the subject invention.

What is claimed is:

1. A transdermal sampling system, comprising:
a microfluidic assembly for retrieving and transferring at least one biomarker transdermally from the skin of a subject, wherein the biomarker is selected from a group of biomarkers consisting of:
a nicotine biomarker, β-hCG, luteinizing hormones, ESAT6; CFP10, Hsp16.3,
marijuana and its derivatives, opiates, narcotics, ethanol, barbiturates, pyridinoline crosslinks, Calcitonin, FSH, estrogen, vitamin D, calcium, glial fibrillary acid protein (GFAP), microtubule-associated protein tau, SNTF-calpain cleaved N terminal fragment proteins, estradiol, P4-progesterone, troponin, lactate, CO₂ levels, and pH levels; and
a transdermal detector for identifying and quantifying the at least one biomarker.

2. A transdermal sampling system, comprising a microfluidic assembly in the form of a patch as recited in the description.
